(19) United States
(12) Patent Application Publication
(10) Pub. No.: US 2020/0199062 A1
(43) Pub. Date: Jun. 25, 2020

(54) METHOD AND SYSTEM FOR SEPARATING ONE OR MORE AMINO ACIDS FROM A WHOLE STILLAGE BYPRODUCT PRODUCED IN A CORN DRY MILLING PROCESS

(71) Applicant: Fluid Quip Technologies, LLC, Springfield, OH (US)

(72) Inventors: Michael Franko, Denver, CO (US); Neal Jakel, Cedar Rapids, IA (US); John Kwik, Bellbrook, OH (US)

(21) Appl. No.: 16/500,593

(22) PCT Filed: May 8, 2018
(86) PCT No.: PCT/US2018/031592

§ 371 Date: Oct. 3, 2019

Related U.S. Application Data
(60) Provisional application No. 62/515,634, filed on Jun. 6, 2017.

(51) Int. Cl. C07C 227/40; C07K 14/425; C07K 1/36; C07K 1/34; C07C 227/44; C07C 319/28; C07D 209/26; A23K 10/38; A23K 20/142; A23L 33/175; C12F 3/10

(52) U.S. Cl. CPC C07C 227/40; C07K 14/425; C07K 1/36; C07K 1/34; C07C 227/44; A23V 2002/00; C07D 209/26; A23K 10/38; A23K 20/142; A23L 33/175; C12F 3/10; C07C 319/28

(57) ABSTRACT

A method for separating one or more amino acids from a whole stillage byproduct produced in a corn dry milling process for making alcohol and system therefore is disclosed. In one embodiment, the method includes separating the whole stillage byproduct into an insoluble solids portion and a thin stillage portion, which include protein and amino acids. Thereafter, the thin stillage portion can be separated into a water soluble solids portion and a protein and amino acids portion. The resulting protein and amino acids portion can be separated into an amino acid(s) portion and a protein portion, both of which may be optionally dried, utilizing microfiltration, ultrafiltration, electrophoresis techniques/devices, or combinations thereof. The amino acid(s) in the resulting amino acid(s) portion may be further processed to be sold and/or used as or in, for example, nutritional supplements, flavor enhancers, animal feed additives, pharmaceuticals, industrial usage, fertilizers, herbicides, or as a feed source for other processing systems.

### TECHNICAL FIELD

The present invention relates generally to corn dry milling, and more specifically, to a method and system for separating one or more amino acids from a whole stillage byproduct produced in a corn (or similar carbohydrate-containing grain) dry milling process for making alcohol, such as ethanol, and/or other biofuels/biochemicals.

### BACKGROUND

Corn or similar starch-containing grains contain not only starch but also protein, individual amino acids, fiber, oil, and minerals. The corn kernel is typically divided up into three main components. The outer protective layer or pericarp, the endosperm, which is the bulk of the kernel material, and the germ or oil-bearing portion of the kernel. The pericarp contains mostly fiber with a small portion of oil bound in the aleurone layer near the outer seed coat. The endosperm contains horny and floury starch components as well as protein. The protein is mainly storage protein or zein protein. The germ contains the bulk of the kernel oil within oil sacks as well as some proteins and key limiting amino acids, sugar, and minerals. More details around the kernel morphology and component constituents can be found in Corn: Chemistry and Technology, 2nd Addition, which is expressly incorporated by reference herein in its entirety.

The amino acids within the kernel of corn are bound together in the form of protein molecules. The germ or embryo typically contains a higher amount of key limiting amino acids compared to the endosperm or storage proteins. There are types of protein complexes contained in the germ fraction, such as albumins and globulins. Additionally, the endosperm contains glutelins and zein protein complexes also known as storage proteins. Within these storage proteins are the individual amino acids. There are 20 known amino acids that naturally occur in corn. Amino acids are classified several ways, but most common is essential and non-essential. Essential amino acids are those that animals or humans must take in for survival. The typical total protein content of corn is about 9% on a dry basis but can vary from 6 to 12% depending on the type of corn and where the corn was grown.

Wet mill corn processing plants convert corn grain into several different natural co-products, such as germ (for oil extraction), gluten feed (high fiber animal feed), gluten meal (high protein animal feed), and starch-based products, including ethanol, high fructose corn syrup, or food and industrial starch. However, because constructing wet milling plants is complex and capital-intensive, almost all new plants built today are dry milling plants.

Dry milling plants generally convert corn into only two products, i.e., ethanol and distiller's grains with solubles. A typical corn dry mill process consists of four major steps: grain handling and milling, liquefaction and saccharification, fermentation, and co-product recovery. Grain handling and milling is the step in which the corn is brought into the plant and ground to promote better starch to glucose conversion. Liquefaction and saccharification is where the starch is converted into glucose. Fermentation is the process of yeast or bacterica, such as clostridia, converting glucose into a biofuel or a biochemical, such as ethanol. Co-product recovery is the step in which the ethanol and corn by-products are purified and made market ready. There are many known chemical and biological conversion processes known in the art that utilize yeast, bacteria, or the like to convert glucose to other biofuels and biochemical components like ethanol, for example.

The recovery of alcohol, e.g., butanol, ethanol (a natural co-product), etc., and other similar compounds, generally begins with the beer being sent to a distillation system. With distillation, ethanol is typically separated from the rest of the beer through a set of stepwise vaporization and condensation. The beer less the alcohol extracted through distillation is known as whole stillage, which contains a slurry of the spent grains including corn protein, fiber, oil, minerals and sugars. This byproduct is too diluted to be of much value at this point and is further processed to provide the distiller's grains with soluble product.

In typical processing, when the whole stillage leaves the distillation system, it is generally subjected to a decanter centrifuge to separate insoluble solids or “wet cake”, which includes fiber, from the liquid or “thin stillage”, which includes, e.g., nitrogen sources (protein) and sulfur sources (amino acids). After separation, the thin stillage moves to evaporators to boil away moisture, leaving a thick syrup that contains the soluble (dissolved) solids. The concentrated syrup is typically mixed with the wet cake, and the mixture may be sold to beef and dairy feedlots as distillers wet grain with solubles (DWGS). Alternatively, the wet cake and concentrated syrup mixture may be dried in a drying process and sold as distillers dried grain with solubles (DDGS). The resulting DDGS generally has a crude protein content of about 32% and is an especially useful feed for cattle and other ruminants due to its by-pass protein content. The resulting product is a natural product.

While DDGS and DWGS provide a critical secondary revenue stream that offsets a portion of the overall ethanol production cost, it would be beneficial to provide a method and system where a backend stream(s) in the corn dry milling process can be utilized to produce one or more other products, such as fertilizers or herbicide, that can provide other or additional revenue.

### SUMMARY

The present invention relates to a method and system for separating one or more amino acids from a whole stillage byproduct produced in a corn dry-milling process for making alcohol, such as ethanol, or other biofuels and/or biochemicals. The corn or other similar carbohydrate materials such as wheat, barley, tritical, sorghum, tapioca, cassava, potato and other grain include amino acids that can be eventually recovered therefrom as a product of the biofuel and/or biochemical dry milling process.

Amino acids are organic compounds that contain amine and carboxylic acid functional groups, and a side chain specific to each amino acid. The key elements of an amino acid are carbon, hydrogen, oxygen, and nitrogen, and in some cases sulfur. Because of their biological significance, amino acids are commonly used in nutritional supplements, food technology (e.g., human food applications, such as flavor enhancers, or animal feed additives), pharmaceuticals, and fertilizers. For example, along with monosodium glutamate (MSG), alanine, aspartate, and arginine are all used to improve the flavor of food. Further, lysine, methionine, threonine, tryptophan, and others improve the nutritional quality of animal feeds by supplying essential amino acids that may be in low abundance in grain. For example, using 0.5% lysine in animal feed improves the quality of the feed as much as adding 20% soybean meal. In addition, by limiting the added amino acid supplements to those required by the animal, some of the excess ammonia made via deamination reactions that is normally excreted to the environment is eliminated. Additionally, amino acids, such as glycine and threonine, are used as precursors for the synthesis of herbicides. Moreover, industrial and pharmaceutical applications of amino acids include the production of biodegradable plastics, chiral catalysts, and drugs.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a flow diagram of a prior art dry milling process and system for producing ethanol and distiller's grains with solubles;

FIG. 2 is a flow diagram of a method and system for separating one or more amino acids from a whole stillage byproduct produced via a corn dry milling process for making alcohol, e.g., ethanol, in accordance with an embodiment of the invention; and

FIG. 3 is a flow diagram of a method and system for separating one or more amino acids from a whole stillage byproduct produced via a corn dry milling process for making alcohol, e.g., ethanol, in accordance with another embodiment of the invention.

### DETAILED DESCRIPTION OF SPECIFIC EMBODIMENTS

The present invention is directed to a method and system for separating one or more amino acids from a whole stillage byproduct produced in a corn dry milling process for making a biofuel, e.g., ethanol, or a biochemical, e.g., lactic acid.

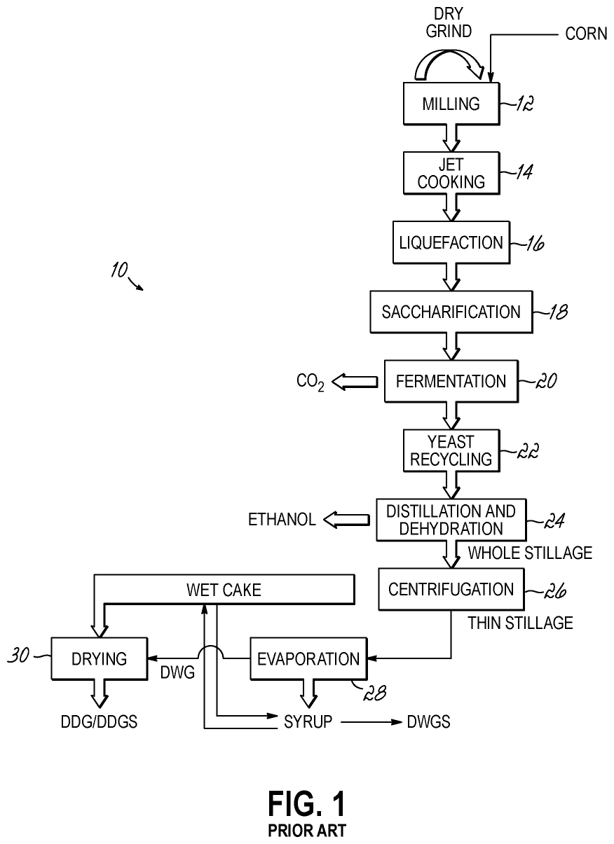

FIG. 1 is a flow diagram of a prior art corn dry milling process for producing alcohol, such process is fully discussed in U.S. Pat. No. 8,778,433, entitled “Methods for producing a high protein corn meal from a whole stillage byproduct”. A significant portion of alcohol, e.g., ethanol, in the United States is produced from dry milling processes, which convert corn into two products, namely ethanol and distiller's grains with solubles. Although virtually any type and quality of grain, such as but not limited to sorghum, wheat, triticale, barley, rye, tapioca, cassava, potato, and other starch containing grains can be used to produce ethanol, the feedstock for this process is typically corn referred to as “No. 2 Yellow Dent Corn.”

With specific reference to FIG. 1, a typical corn dry milling process 10 begins with a milling step 12 in which dried whole corn kernels are passed through hammer mills to grind them into meal or a fine powder. The ground meal is mixed with water to create a slurry, and a commercial enzyme such as alpha-amylase is added. This slurry is then typically pH adjusted and heated in a pressurized jet cooking process 14 to solubilize the starch in the ground meal. This is followed by a liquefaction step 16 at which point additional alpha-amylase may be added. The alpha-amylase hydrolyzes the gelatinized starch into maltodextrins and oligosaccharides to produce a liquefied mash or slurry.

This can be followed by separate saccharification and fermentation steps, 18 and 20, respectively, which may include a pH and temperature adjustment from the separate liquefaction step, although in most commercial dry milling ethanol processes, saccharification and fermentation occur simultaneously. In the saccharification step 18, the liquefied mash is cooled and a commercial enzyme, such as gluco-amylase, is added to hydrolyze the maltodextrins and short-chained oligosaccharides into single glucose sugar molecules. In the fermentation step 20, a common strain of yeast (Saccharomyces cerevisae) is added to metabolize the glucose sugars into ethanol and CO₂. Other fermentation agents such as bacteria and clustridia can be utilized. Upon completion, the fermentation mash (“beer”) will contain about 17% to 18% ethanol (volume/volume basis), plus soluble and insoluble solids from all the remaining grain components, including fiber, protein, minerals, and oil, for example. Yeast can optionally be recycled in a yeast recycling step 22. In some instances, the CO₂ is recovered and sold as a commodity product.

Subsequent to the fermentation step 20 is a distillation and dehydration step 24 in which the beer is pumped into distillation columns where it is boiled to vaporize the ethanol. The ethanol vapor after exiting the top of the distillation column is condensed and liquid alcohol (in this instance, ethanol) is about 95% purity (190 proof). The 190 proof ethanol can then go through a molecular sieve dehydration column or a membrane separation unit or similar dehydration system, which removes the remaining residual water from the ethanol, to yield a final product of essentially 100% ethanol (199.5 proof).

Finally, a centrifugation step 26 involves centrifuging, via a decanter centrifuge, the residuals or whole stillage leftover from distillation so as to separate the insoluble solids portion or “wet cake”, which includes fiber, from the liquid portion or “thin stillage” portion, which includes protein, amino acids, oil, etc. Next, the thin stillage portion enters evaporators in an evaporation step 28 in order to boil away moisture thereby leaving a thick syrup, which contains the soluble (dissolved) solids as well as protein and oil. This concentrated syrup is typically referred to as corn condensed distillers soluble and is mixed with the centrifuged wet cake then sold to beef and dairy feedlots as distillers wet grain with solubles (DWGS). The wet cake and concentrated syrup mixture may be further dried in a drying step 30 and sold as distillers dried grain with solubles (DDGS) to dairy and beef feedlots and/or the monogastric markets. The distiller's grains with solubles co-product provides a critical secondary revenue stream that offsets a portion of the overall ethanol production cost.

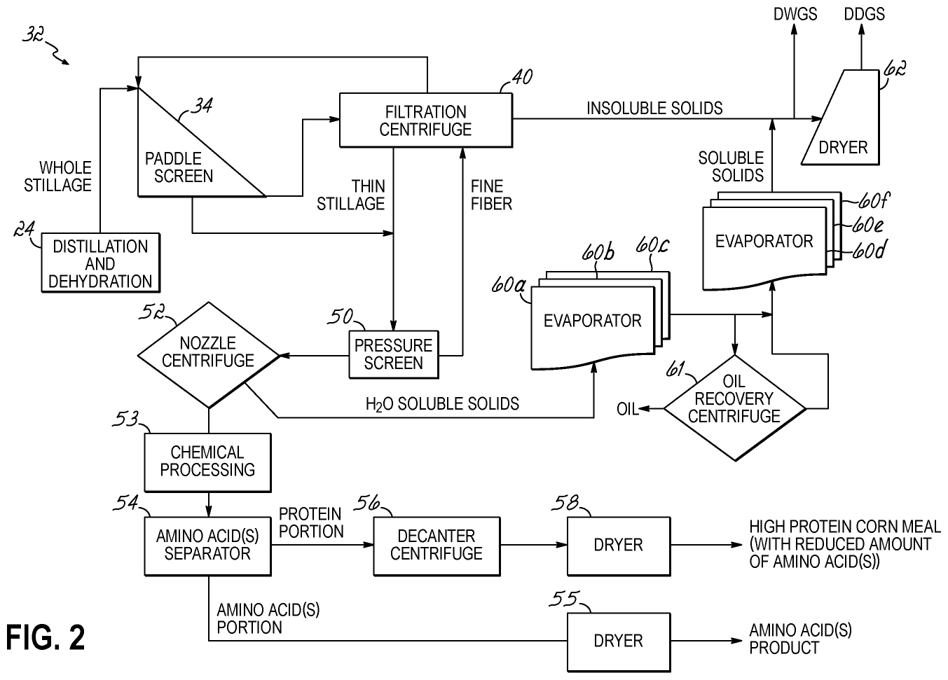

In accordance with the present invention, FIG. 2 schematically illustrates an embodiment of a method and system for separating one or more amino acids, collectively numeral 32, from the whole stillage byproduct produced in a typical corn dry-milling process 10, like that just described in FIG. 1. While a typical whole stillage byproduct is utilized here, it should be understood that the whole stillage from any corn (or similar carbohydrate-containing grain) dry milling process may be utilized with the same or similar results. Again, the whole stillage byproduct contains a slurry of soluble and insoluble solids, i.e., the spent grains from the distillation and dehydration step 24, which can include amino acids, protein, fiber, minerals, and free oil, for example, that can be processed in accordance with embodiments of this invention to separate one or more amino acids. The separated amino acid(s) may be further processed to be sold and/or used as or in, for example, nutritional supplements, flavor enhancers, animal feed additives, fertilizers, herbicides, industrial applications, pharmaceuticals, and other products or as a feed source for other processing systems.

With continuing reference to FIG. 2, the whole stillage byproduct can be piped from the typical corn dry milling distillation and dehydration step 24 and subjected to an optional paddle screen 34. The optional paddle screen 34 is situated before a 2 40, which is further discussed below, so as to aid ultimately in separation of the insoluble solids portion, e.g., fiber, from the thin stillage portion by initially filtering out desirable amounts of water, amino acids (e.g., lysine and tryptophan), protein, and, incidentally, small fiber fines from the whole stillage byproduct. This initial screening can help reduce the resulting load on the subsequent filtration centrifuge 40. The resulting thrus (centrate) from the paddle screen 34 eventually joins with the thin stillage underflow from the filtration centrifuge 40, as will be discussed in greater detail below.

To filter the whole stillage byproduct, the optional paddle screen 34 can include screen openings of no greater than about 500 microns. In another example, the paddle screen 34 can include openings therein of no greater than about 400 microns. In yet another example, the openings therein are no greater than about 300 microns. In yet another example, the paddle screen 34 can include openings therein of no greater than about 150 microns and yet another example, the paddle screen 34 can include openings therein of no greater than about 50 microns. It should be understood that these values are exemplary and that those of ordinary skill in the art will recognize how to determine the size of the openings to achieve the desired filtration. In one example, the optional paddle screen 34 is a standard type paddle screen as is known in the art. One such suitable paddle screen 34 is the FQ-PS32 available from Fluid-Quip, Inc. of Springfield, Ohio. It should be understood that the optional paddle screen 34 may be replaced with other types of pre-concentration devices, e.g., a standard pressure screen, conic centrifuge, cyclone, or hydroclone, which can perform the desired filtration or preconcentration function. One such suitable pressure screen is the PS-Triple available from Fluid-Quip, Inc. of Springfield, Ohio. In addition, although a single paddle screen 34 is depicted, it should be understood that a plurality of screens 34 may be situated in-line and utilized for filtering the whole stillage byproduct.

The whole stillage from the distillation and dehydration step 24, if the optional paddle screen 34 is not present, or the cake (solids) from the optional paddle screen 34 is sent to the filtration centrifuge 40 whereat the whole stillage byproduct or cake is separated into the insoluble solids portion, which includes fiber, and the thin stillage portion, which includes amino acids (e.g., lysine and tryptophan), protein, free oil, etc. One such suitable filtration centrifuge is described in Lee et al., U.S. Pat. No. 8,813,973 entitled “Apparatus and Method for Filtering a Material from a Liquid Medium”, the contents of which are expressly incorporated by reference herein in its entirety. The filtration centrifuge 40 may be configured to perform both the initial filtering (sometimes referred to as a pre-concentration) of the whole stillage byproduct and washing of the fiber so as to clean the fiber and remove the amino acids, protein, free oil, and other components that remain associated with the fiber after the initial filtration or pre-concentration.

With respect to the filtration centrifuge 40, the washing of the fiber may include a washing cycle, wherein the fiber is mixed and rinsed in wash water, followed by a de-watering cycle, wherein the wash water is separated from the fiber. The washing of the fiber may include multiple rinsing/de-watering cycles. Additionally, a counter current washing technique may be employed to save wash water usage. After washing the fiber, but before the fiber exits the centrifuge, the fiber may go through an enhanced de-watering stage, a compaction stage, and/or an air dry stage to further de-water or dry the fiber. This may reduce the dryer capacity or eliminate the dryer altogether. Eventually, the washed and filtered fiber exits the filtration centrifuge 40 so that the fiber can be further processed, as discussed further below to result in a desired product, such as DWGS or DDGS. In one example, the fiber can be transported to a remote site for further processing. Moreover, any separated out portion of slurry from the fiber, e.g., water, amino acids, protein, free oil, wash water, etc., which occurs via screening, is collected to define the thin stillage, then transported and further processed as described below. Optionally, a portion of the slurry and/or wash water may be piped back to the optional paddle screen 34 for further reprocessing. The filtration centrifuge 40 may provide the filtered material at a water concentration of between about 55% and about 75% water, which is a significant reduction compared to conventional filtration systems.

With continuing reference to FIG. 2, although a single filtration centrifuge 40 is depicted, it should be understood that a plurality of filtration centrifuges 40, either in parallel or series, may be situated in-line and utilized for separating the whole stillage byproduct into its insoluble solids portion (fiber) and thin stillage portion. And in an alternate embodiment, it is contemplated that the filtration centrifuge 40 can be replaced by a standard pressure screen, decanter centrifuge, a paddle screen, or other like devices as are known in the art to separate the whole stillage byproduct into the insoluble solids portion and thin stillage portion. One such suitable pressure screen is the PS-Triple available from Fluid-Quip, Inc. of Springfield, Ohio. One such suitable decanter centrifuge is the NX-944HS available from Alfa Laval of Lund, Sweden. And one such suitable paddle screen is the FQ-PS32 available from Fluid-Quip, Inc. of Springfield, Ohio. To further enhance the separation of non-protein/amino acid components from the remaining kernel components, a flocculent can optionally be added to the whole stillage prior to a first separation step. A flocculent may help bind fiber and other kernel components together, making separation of those particles more efficient, thus resulting in improved protein and amino acid recovery downstream.

As further shown in FIG. 2, the thin stillage centrate (underflow) from the filtration centrifuge 40 is piped to join up with the thrus (underflow) from the optional paddle screen 34 prior to or at an optional standard pressure screen 50, as is known in the art, to further aid in separation of any fine fiber from the thin stillage portion. If the optional paddle screen 34 is not present, the thin stillage underflow from the filtration centrifuge 40 is sent directly to optional pressure screen 50. Prior to being subjected to the optional pressure screen 50, the thin stillage can include a number of the amino acids and protein contained within the kernel of corn. The protein content within this stream ranges from 24.8% to 33.2% and solids content within this stream ranges from 5% to 15%.

Fiber having a size less than that of the screen of the filtration centrifuge 40 and/or optional paddle screen 34 may pass through and to subsequent steps of the corn dry milling process. At the pressure screen 50, the separated fine fiber can be separated from the thin stillage and piped back to the filtration centrifuge 40 or similar unit operations whereat the fine fiber may be filtered out. One such suitable pressure screen 50 is the PS-Triple available from Fluid-Quip, Inc. of Springfield, Ohio. In an alternate embodiment, the optional pressure screen 50 may be replaced with a standard paddle screen or decanter centrifuge, as are mentioned above, or other like device, such as a filtration centrifuge, to aid in separation of the fine fiber from the thin stillage portion. In addition, although a single pressure screen 50 is depicted, it should be understood that a plurality of pressure screens 50, either in parallel or series, may be situated in-line and utilized for filtering the thin stillage underflow.

After the optional pressure screen 50, the remaining thin stillage portion can include a total protein content, as measured in a spin tube, of about 10 ml/50 ml of liquid sample. Additional protein and amino acids that are water soluble (all are, they just vary in solubility) will be in the supernate of the spun sample (water portion on top of the solid portion of the spun sample). The amount of protein in the spun sample can vary due to a number of factors; the value here represents an average. The protein content in the spun sample may be smaller or larger depending on upstream process variations and initial corn kernel protein content. The protein content in a 50 ml spin tube can have a range from about 5 ml up to 25 ml. The remaining thin stillage portion from the optional pressure screen 50 is piped and subjected to a nozzle centrifuge 52, as is known in the art. Alternatively, if the optional pressure screen 50 is not present, the thin stillage centrate can be sent directly to the nozzle centrifuge 52. The nozzle centrifuge 52 can be provided with washing capabilities so that water, or similar aqueous solutions, along with the thin stillage portion, can be supplied to the nozzle centrifuge 52. At this step, the additional water or aqueous solution allows for easier separation of the thin stillage into a protein and amino acids portion given that all amino acids are water soluble (the degree to which amino acids are water soluble does vary for the 20 known amino acids) and a water soluble solids portion. The heavier protein and amino acids portion separates from the lighter water soluble solids portion and is removed as the underflow whereas the lighter water soluble solids portion, which includes free oil and sugars, can be removed as the overflow. One such suitable nozzle centrifuge 52 is the FQC-950 available from Fluid-Quip, Inc. of Springfield, Ohio. In an alternate embodiment, the nozzle centrifuge 52 can be replaced with a standard cyclone apparatus or other like device, as are known in the art, to separate the thin stillage portion into the underflow protein portion and overflow water soluble solids portion. One such suitable cyclone apparatus is the RM-12-688 available from Fluid-Quip, Inc. of Springfield, Ohio.

The underflow protein and amino acids portion from the nozzle centrifuge 52 is then optionally piped and subjected to a chemical processing system 53 to assist in separating the amino acid(s) from a protein complex, such as by extracting a certain amino acid(s) from the concentrate underflow stream. In particular, amino acids can be separated from the protein complex that they are typically bound to by several different techniques. In one embodiment, the chemical processing system 53 may include separating the amino acid(s) from a protein complex via acid hydrolysis, which is typically achieved by the addition of a strong acid, such as sulfuric acid and/or phosphoric acid. In another embodiment, the amino acid(s) can be separated from a protein complex by the addition of an alkaline solution (e.g., caustic soda or similar). In another embodiment, the chemical processing system 53 may include extracting a specific amino acid(s) based on solubility characteristics. For example, certain amino acids are more water soluble, while some amino acids and protein complexes are more soluble and separate better in the presence of an alcohol, such as ethanol or isopropanol or the like. In yet another embodiment, the chemical processing system 53 may include the catalytic reaction of the proteins to further aid in the separation of individual amino acids from a protein complex. Additionally, certain organic and/or inorganic solvents can be utilized to remove certain amino acids from a protein complex. The amount and concentration of the above mentioned chemical processing aides can vary greatly depending on the final amino acid concentration and individual amino acids, as desired. Further, the chemical processing system 53 can consist of a single processing step or several processing steps.

The optionally processed protein and amino acids portion from the chemical processing system 53 can then be piped and subjected to an amino acid(s) separator 54. If the optional chemical processing system 53 is not present, then the underflow protein and amino acids portion from the nozzle centrifuge 52 is piped and subjected to the amino acid(s) separator 54. At the amino acid(s) separator 54, one or more amino acids can be separated from the underflow protein and amino acids portion resulting in a separate amino acid(s) portion and protein portion. The amino acid(s) separator 54 can separate the amino acid(s) from the underflow protein portion using one or more of the following systems/technologies: membrane filtration, such as microfiltration or ultrafiltration, electrophoresis techniques, ion adsorption, or combinations thereof. To that end, the amino acid(s) separator 54 can define or include microfiltration, ultrafiltration, electrophoresis devices, ion adsorption, or combinations thereof. In one example, one or more specific amino acids may be selectively targeted to provide a desired amino acid portion (or product) and/or a desired protein portion (or product).

Concerning microfiltration devices, microfiltration membranes typically include polymer, ceramic, paper, or metal membrane disc or pleated cartridge filters generally rated in the 0.1 to 2 micron range and that generally operate in the 1 to 25 psig pressure range. One such suitable microfiltration amino acid(s) separator is the PURON PLUS MBS system provided by Koch Membrane Systems of Wichita, Kans. Concerning ultrafiltration device, ultrafiltration is a crossflow process generally rated in the 10 angstrom to 0.1 micron range and that generally operates in the 10 to 100 psig range. One such suitable ultrafiltration amino acid(s) separator is the HFK Series Ultrafiltration provided by Koch Membrane Systems of Wichita, Kans. Concerning electrophoresis devices, ions, molecules, and particles with charge carry current in solutions when an electromagnetic field is imposed. Thus, in an embodiment where the amino acid(s) separator 54 utilizes electrophoresis, the protein molecules and the amino acid molecules, which may be charged, will tend to move toward the electrode of opposite charge, thus separating. One such suitable electrophoretic amino acids separator is the SE600 from AA Hoefer. In addition, although a single amino acid(s) separator 54 is depicted, it should be understood that a plurality of amino acid(s) separators 54 either in series or parallel may be situated in-line and utilized for separating the amino acid(s) portion from the protein and amino acids portion.

After separation, the amino acid(s) portion may be optionally stabilized by a stabilizer, which may be added to the amino acid portion. In one example, the stabilizer may be a salt, a carbohydrate, cellulose, a dextrin component, or a combination thereof. For example, a salt or salt component may be added to the amino acid(s) portion, such as calcium, sodium, and the like, in concentrations ranging from 0.001% up to 1% or greater of the solution. The final solution concentration is dependent upon the final amount and type of specific amino acid(s) that is desired to be separated and stabilized. Amino acids can readily degrade once separated from a protein complex due to oxygen and temperature. To help stabilize and prevent the degradation of the amino acids, salts are sometimes added to the amino acid mixture. The amino acid(s) portion from the amino acid(s) separator 54 can be further optionally dried and/or crystallized, such as by being sent to a dryer 55, e.g., a spray dryer, and/or a crystallizer, as is known in the art. In another embodiment, the amino acid(s) portion can be subjected to vacuum filtration or other dewatering and drying or crystallizing methods as are known in the art. The type and concentration of the amino acid(s) present in the final amino acid(s) portion may vary based on the carbohydrate-containing grain source and the specific application. In one example, the amino acid(s) portion or product can include one or more of lysine, methionine, tryptophan, threonine, valine, isoleucine, leucine, phenylalanine, histidine, arginine, cysteine, glutamic acid, aspartic acid, serine, alanine, tyrosine, or proline. In another example, the amino acid(s) portion includes one or more of lysine, methionine, cysteine, threonine, tryptophan, valine, isoleucine, or leucine. In another example, the amino acid(s) portion or product includes one or more of lysine, methionine, tryptophan, or threonine. The concentration of the amino acid(s) in the amino acid(s) portion or product can be higher than the concentration in the protein and amino acids portion. In one example, the amino acid(s) will have a concentration that is 1% to 80% greater in the amino acid(s) portion or product. In another example, the amino acid(s) will have a concentration that is 20% to 60% greater. As indicated above, the resulting amino acid(s) portion or product may be sold and/or used as or in nutritional supplements, flavor enhancers, animal feed additives, pharmaceuticals, industrial usage, fertilizers, or herbicides for example.

The resulting protein portion from the amino acid(s) separator 54 can then be piped and subjected to a decanter centrifuge 56. At the decanter centrifuge 56, the protein portion is dewatered to provide a dewatered protein portion. The decanter centrifuge 56 is standard and known in the art. One such suitable decanter centrifuge 56 is the NX-944HS available from Alfa Laval of Lund, Sweden. In addition, although a single decanter centrifuge 56 is depicted, it should be understood that a plurality of decanter centrifuges 56 may be situated in-line, either in series or parallel, and utilized for filtering the thin stillage underflow. In an alternate embodiment, the decanter centrifuge 56 may be replaced with a standard filter press or rotary vacuum, or other like device, as are known in the art, to dewater the thin stillage portion. A water portion or filtrate from the decanter centrifuge 56 may be recycled back, for example, to liquefaction step 16 or fermentation step 20 for reuse in the dry milling process.

The dewatered protein portion from the decanter centrifuge 56 can be further optionally dried, such as by being sent to a dryer 58, e.g., a spray dryer, or a crystallizer, as is known in the art. In another embodiment, the dewatered protein portion can be subjected to vacuum filtration or other dewatering and drying methods, as are known in the art. The final dried protein portion or product defines a high protein corn meal, which can have a reduced amount of one or more amino acids due to the prior separation. In one example, the final dried protein portion or product defines a high protein corn meal that can include, for example, the amino acids and their concentrations listed in Table 1 below.

TABLE 1

| | Weight | | Weight |
|---|---|---|---|
| Amino Acid | Percentage | Amino Acid | Percentage |
| Lysine | 2.09 | Arginine | 2.34 |
| Methionine | 1.01 | Cysteine | 0.99 |
| Tryptophan | 0.45 | Glutamic acid | 7.51 |
| Threonine | 2.10 | Aspartic acid | 3.55 |
| Valine | 2.73 | Serine | 2.27 |
| Isoleucine | 2.06 | Glycine | 1.93 |
| Leucine | 5.37 | Alanine | 3.50 |
| Phenylalanine | 2.47 | Tyrosine | 1.91 |
| Histidine | 1.38 | Proline | 3.55 |

In another example, the final protein portion or product can include one or more of lysine, methionine, tryptophan, threonine, valine, isoleucine, leucine, phenylalanine, histidine, arginine, cysteine, glutamic acid, aspartic acid, serine, alanine, tyrosine, or proline. In another example, the final protein portion or product can be devoid of one or more of lysine, methionine, tryptophan, threonine, valine, isoleucine, leucine, phenylalanine, histidine, arginine, cysteine, glutamic acid, aspartic acid, serine, alanine, tyrosine, or proline. In another example, the final protein portion or product can be devoid of one or more of lysine, methionine, tryptophan, or threonine. The concentration of the amino acid(s) in the protein portion or product can be higher than its concentration in the protein and amino acids portion. In one example, the amino acid(s) will have a concentration that is 1% to 80% greater in the protein portion or product. In another example, the amino acid(s) will have a concentration that is 10% to 50% greater. In another example, one or more amino acids can have a lower concentration than listed in Table 1. The high protein corn meal may be sold as pig feed, chicken feed, aqua food uses, or have other uses, including pharmacueitcal and/or chemical usage, for example. The resulting high protein corn meal may be sold at a much higher cost per ton than DDGS or DWGS.

Returning now to the separated water soluble solids portion or filtrate from the nozzle centrifuge 52, which includes free oil as well as minerals, sugar and soluble proteins, the separated water soluble solids portion may be recycled back, for example, to the liquefaction step 16 or the fermentation step 20 for reuse in the dry milling process. Additionally or alternatively, as shown in FIG. 2, the water soluble solids portion can be piped from the nozzle centrifuge 52 and subjected to a set of three evaporators 60a, 60b, and 60c, as are known in the art, to begin separating the soluble solids from the water soluble solids portion. The evaporators 60a-c evaporate the liquid portion of the water soluble solids portion. Thereafter, the water soluble solids portion can be piped and subjected to an optional oil recovery centrifuge 61, as is known in the art, so that oil can be removed therefrom. One such suitable oil recovery centrifuge 261 is the ORPX 617 available from Alfa Laval of Lund, Sweden. In one example, the final recovered oil product can include between about 30 wt % to about 60 wt % of the total corn oil in the corn. In comparison to typical oil recovery in a standard dry milling process, oil recovery centrifuge 61 can function at a higher capacity because the water soluble solids portion, which is subjected to the oil recovery centrifuge 61, includes less liquid and less protein and fiber than normal.

The remainder of the water soluble solids portion can be piped and subjected to another set of three evaporators 60d, 60e, and 60f whereat the liquid portion is further evaporated from the water soluble solids portion to ultimately yield a soluble solids portion. While the water soluble solids portion is subjected to two sets of three evaporators 60a-c, 60d-f, it should be understood that the number of evaporators and sets thereof can be varied, i.e., can be more or less, from that shown depending on the particular application and result desired. Additionally, this soluble solids stream can be further processed as a raw material feed source, such as for a bio-digester to produce biofuels and/or biochemicals, an algae feed source, or further processed via fermentation, for example, to yield a high protein nutrient feed.

The resulting soluble solids portion may be combined with the insoluble solids portion, e.g., fiber, received from the filtration centrifuge 40 to provide distillers wet grains with soluble (DWGS), which may be further dried by a drier 62, as is known in the art, to provide distillers dry grains with solubles (DDGS), both of which can be sold to dairy or beef feedlots, monogastric markets for monogastric animals, or as pet food or aquaculture. In another example, the soluble solids portion may be used as a natural fertilizer. In another example, the soluble solids portion may be used as a raw material feed source for conversion to simple sugar, which can be further converted to biofuel or used in other biochemical processes.

Accordingly, in this dry-milling process, neither the DDGS nor DWGS receive the typical concentrated syrup from the evaporators 60. And, despite the lower protein content, the DDGS and DWGS may still be sold to beef and dairy feedlots as cattle feed or other animal feed markets.

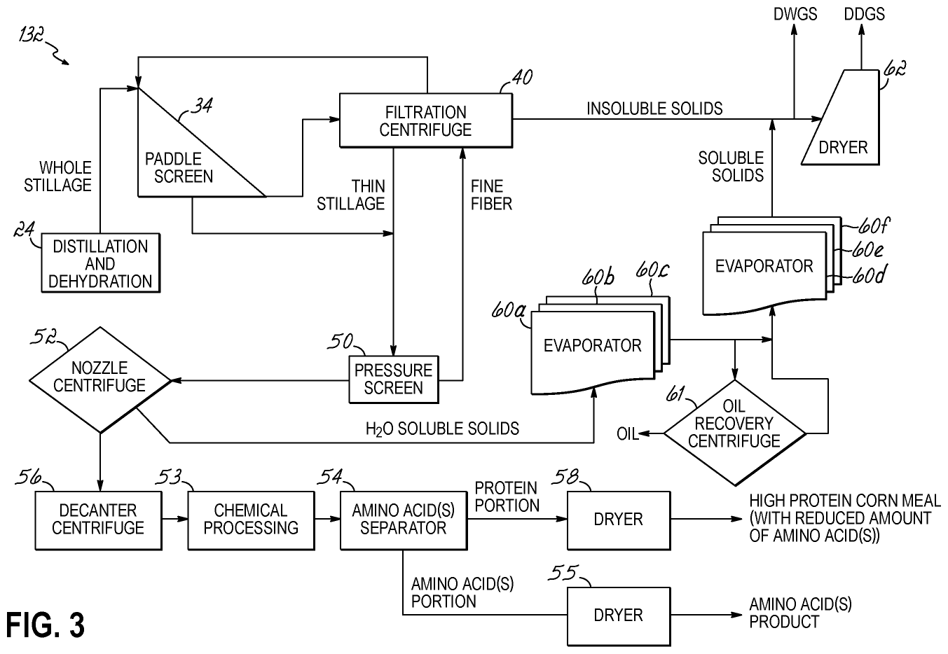

With reference now to FIG. 3, another embodiment of a method and system for separating one or more amino acids, collectively numeral 132, from a whole stillage byproduct produced in a typical corn dry-milling process 10, is shown. In this embodiment, the protein and amino acids portion resulting from the nozzle centrifuge 52, in contrast to the method and system 32 shown in FIG. 2, is piped and subjected to the decanter centrifuge 56 before being subjected to the optional chemical processing system 53 and the amino acid(s) separator 54. As indicated, at the decanter centrifuge 56, the protein and amino acids portion is dewatered to provide a dewatered protein and amino acids portion. In addition, although a single decanter centrifuge 56 is depicted, it should be understood that a plurality of decanter centrifuges 56 may be situated in-line, either in parallel or series, and utilized for filtering the protein and amino acids portion or the thin stillage underflow. In an alternate embodiment, the decanter centrifuge 56 may be replaced with a standard filter press or rotary vacuum, or other like device, as are known in the art, to dewater the thin stillage portion. A water portion or filtrate from the decanter centrifuge 56 may be recycled back, for example, to the liquefaction step 16 or the fermentation step 20 or other steps within the biofuels conversion process for reuse in the dry-milling process.

The dewatered protein and amino acids portion from the decanter centrifuge 56 can be piped and subjected to the optional chemical processing system 53 and/or piped and subjected to the amino acid(s) separator 54. As indicated above, at the chemical processing system 53, the amino acid(s) may be separated from a protein complex or may be extracted from the concentrate underflow stream. At the amino acid(s) separator 54, an amino acid portion can be separated from the dewatered protein and amino acids portion resulting in a separate amino acid(s) portion and protein portion. As indicated above, a number of separation techniques may be used in the amino acid(s) separator 54. For example, the amino acid(s) separator 54 may separate one or more amino acids from the protein using membrane filtration, such as microfiltration or ultrafiltration, or electrophoresis techniques, or combinations thereof. To that end, the amino acid(s) separator 54 can define or include microfiltration, ultrafiltration, electrophoresis devices, ion adsorption, or combinations thereof. The amino acid(s) portion from the amino acid(s) separator 54 may be stabilized. The amino acid(s) portion also can be further optionally dried and/or crystallized, such as by being sent to dryer 55, e.g., a spray dryer, and/or crystallizer, as is known in the art. In another embodiment, the amino acid(s) portion can be subjected to vacuum filtration or other drying or crystallizing methods, as are known in the art. As indicated above, the resulting amino acid(s) portion or product may be sold and/or used as or in nutritional supplements, flavor enhancers, animal feed additives, industrial usage, pharmaceuticals, a feed source for a chemical conversion process, fertilizers, or herbicide, for example.

The dewatered protein portion resulting from the amino acid(s) separator 54 can be further optionally dried, such as by being sent to dryer 58, e.g., a spray dryer, or a crystallizer, as is known in the art. In another embodiment, the dewatered protein portion can be subjected to vacuum filtration or other drying methods, as are known in the art. The final dried protein portion or product defines a high protein corn meal. As indicated, the high protein corn meal may be sold as animal feed at a much higher cost per ton than DDGS or DWGS.

While the present invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. For example, while embodiments of the method and system 32, 132 herein focus on separating an amino acid(s) portion from a stream coming from the nozzle centrifuge 52, there are other locations in the method and system 32, 132 where amino acids can be separated from a stream, such as via an amino acid(s) separator 54, which may further optionally include an amino acid chemical processing system 53. In one example, one or more amino acids may be separated from the stream from the optional pressure screen 50 or from the water soluble solids portion from the nozzle centrifuge 52. Thus, the invention in its broader aspects is therefore not limited to the specific details, representative apparatus and method, and illustrative example shown and described. Accordingly, departures may be made from such details without departing from the scope of applicant's general inventive concept.

What is claimed is:

1. A method for separating one or more amino acids from a whole stillage byproduct produced in a starch-containing grain dry milling process for making biofuel and/or a biochemical, the method comprising:
separating the whole stillage byproduct into an insoluble solids portion and a thin stillage portion, including protein, amino acids, and free oil;
separating the thin stillage portion into a water soluble solids portion, including the free oil, and a protein and amino acids portion, including the protein and the amino acids;
separating the protein and amino acids portion into a protein portion and an amino acids portion, which includes one or more of the amino acids; and
recovering the amino acids portion.

2. The method of claim 1, further comprising drying the separated amino acids portion to define an amino acid product.

3. The method of claim 1, further comprising drying the separated protein portion to define a protein product.

4. The method of claim 3, wherein the protein product is a high protein corn meal that includes at least 40 wt % protein on a dry basis.

5. The method of claim 1, wherein separating the protein and amino acids portion into a protein portion and an amino acids portion comprises separating the protein and amino acids portion via membrane filtration, electrophoresis, ion adsorption, or a combination thereof.

6. The method of claim 1, wherein separating the protein and amino acids portion into a protein portion and an amino acids portion comprises separating the protein and amino acids portion via a microfiltration membrane, an ultrafiltration membrane, or a combination thereof.

7. The method of claim 1, further comprising, prior to separating the protein and amino acids portion into a protein portion and an amino acids portion, chemically processing the protein and amino acids portion to aid the separation of the amino acids portion from the protein portion.

8. The method of claim 7, wherein chemically processing the protein and amino acids portion includes adding one of a strong acid, an alkaline solution, an alcohol, or a catalyst to the protein and amino acids portion.

9. The method of claim 1, further comprising stabilizing the separated amino acids portion.

10. The method of claim 9, wherein stabilizing the separated amino acids portion includes adding a stabilizer that includes a salt, a carbohydrate, cellulose, a dextrin component, or a combination thereof to the separated amino acids portion.

11. The method of claim 1, further comprising, prior to separating the protein and amino acids portion into the protein portion and the amino acids portion, dewatering the separated protein and amino acids portion.

12. The method of claim 1, further comprising dewatering the separated protein portion.

13. The method of claim 1, further comprising, after separating the whole stillage byproduct into an insoluble solids portion and a thin stillage portion and before separating the thin stillage portion into a protein portion and a water soluble solids portion, separating fine fiber from the thin stillage portion.

14. The method of claim 13, wherein separating fine fiber from the thin stillage portion comprises separating the fine fiber via a pressure screen, paddle screen, decanter, or filtration centrifuge.

15. The method of claim 1, further comprising separating soluble solids from the water soluble solids portion to provide a soluble solids portion.

16. The method of claim 1, further comprising separating the free oil from the water soluble solids portion to provide an oil portion.

17. The method of claim 1, further comprising processing the separated amino acids to be used as or in animal feed or human food applications.

18. The method of claim 1, wherein the amino acids portion includes one or more of lysine, methionine, cysteine, threonine, tryptophan, valine, isoleucine, or leucine.

19. A system for separating one or more amino acids from a whole stillage byproduct produced in a starch-containing grain dry milling process for making biofuel and/or a biochemical, the system comprising:
a first apparatus that receives the whole stillage byproduct and separates the whole stillage byproduct into an insoluble solids portion and a thin stillage portion, including protein, amino acids, and free oil;
a second apparatus that is situated after the first apparatus, the second apparatus receives the thin stillage and separates the thin stillage portion into a water soluble solids portion, including the free oil, and a protein and amino acids portion, including the protein and the amino acids; and
a third apparatus that is situated after the second apparatus, the third apparatus receives the protein and amino acids portion and separates the protein and amino acids portion into a protein portion and an amino acids portion, which includes one or more of the amino acids.

20. The system of claim 19, further comprising a drying apparatus that is situated after the third apparatus and that dries the amino acids portion received from the third apparatus to define an amino acid product.

21. The system of claim 19, wherein the protein portion defines a high protein corn meal that includes at least 40 wt % protein on a dry basis.

22. The system of claim 19, wherein the first apparatus is selected from a filtration centrifuge, a pressure screen, or a paddle screen and the second apparatus is selected from a nozzle centrifuge or a cyclone apparatus.

23. The system of claim 19, wherein the third apparatus is configured to separate the protein and amino acids portion via membrane filtration, electrophoresis, ion adsorption, or a combination thereof.

24. The system of claim 19, wherein the third apparatus is selected from a microfiltration membrane, an ultrafiltration membrane, or a combination thereof.

25. The system of claim 19, further comprising a dewatering apparatus that is situated after the second apparatus to dewater the protein and amino acids portion or that is situated after the third apparatus to dewater the protein portion.

26. The system of claim 18, further comprising an oil recovery centrifuge to separate the free oil from the water soluble solids portion to provide an oil portion.

27. The system of claim 19, further comprising a drying apparatus that is situated after the third apparatus and that dries the protein portion received from the third apparatus to define a protein product.

28. A system for separating one or more amino acids from a whole stillage byproduct produced in a starch-containing grain dry milling process for making biofuel and/or a biochemical, the system comprising:
a first apparatus that receives the whole stillage byproduct and separates the whole stillage byproduct into an insoluble solids portion and a thin stillage portion, including protein, amino acids, and free oil;
a second apparatus that is situated after the first apparatus, the second apparatus receives the thin stillage and separates the thin stillage portion into a water soluble solids portion, including the free oil, and a protein and amino acids portion, including the protein and the amino acids;
a chemical processing system that is situated after the second apparatus, the chemical processing system receives and chemically processes the protein and amino acids portion to aid in separation of the protein and amino acids portion; and
a third apparatus that is situated after the chemical processing system, the third apparatus receives the chemically processed protein and amino acids portion and separates the protein and amino acids portion into a protein portion and an amino acids portion, which includes one or more of the amino acids.
